(19) United States
(12) Patent Application Publication
(10) Pub. No.: US 2005/0131522 A1
(43) Pub. Date: Jun. 16, 2005

(54) Medical devices and methods of making the same

(76) Inventors: Jonathan S. Stinson, Minneapolis, MN (US); Gary L. Campbell, Maple Grove, MN (US); Steven E. Walak, Natick, MA (US)

Correspondence Address: FISH & RICHARDSON PC, 225 FRANKLIN ST, BOSTON, MA, 02110, US

(21) Appl. No.: 10/732,492

(22) Filed: Dec. 10, 2003

(51) Int. Cl. A61F 2/06

(52) U.S. Cl. 623/1.15; 623/23.7; 623/901

(57) ABSTRACT

Medical devices, such as stents, and methods of making the devices are disclosed. In some embodiments, the devices are made using a laser forming process.

### TECHNICAL FIELD

The invention relates to medical devices, such as stents, and methods of making the devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents and covered stents, sometimes called “stent-grafts”.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

To support a passageway open, endoprostheses are sometimes made of relatively strong materials, such as stainless steel or Nitinol (a nickel-titanium alloy), formed into struts or wires. These materials, however, can be relatively radiolucent. That is, the materials may not be easily visible under X-ray fluoroscopy, which is a technique used to locate and to monitor the endoprostheses during and after delivery. To enhance their visibility (e.g., by increasing their radiopacity), the endoprostheses can be formed with a relatively radiopaque material, such as gold.

### SUMMARY

The invention relates to medical devices, such as stents, and methods of making the medical devices.

In one aspect, the invention features a method of making a medical device using a laser forming process. The laser forming process can be used to tailor the structure and the mechanical, physical, and biocompatibility properties of the device.

In another aspect, the invention features a method of making a medical device. The method includes melting a first material on a substrate, and solidifying the material, wherein the solidified first material forms a portion of the medical device.

Embodiments may include one or more of the following features. The first material is melted by a laser. The laser and/or the substrate is moved relative to each other. The method further includes forming a layer of the first material surrounding the substrate. The method further includes melting a second material on the first material. The method further includes delivering the first material to the substrate. The method further includes separating a portion of the substrate from the first material. The method includes forming the first material into a tubular member, and optionally, forming the tubular member into a stent. The method further includes changing the composition of the first material. The method further includes changing the operating conditions of the laser during melting of the first material. The method further includes placing an elongated second material more radiopaque than the first material on the solidified first material.

The first material can have one or more of the following features. The first material includes an alloy, such as stainless steel. The first material is in the form of a powder, and the method further includes changing the size of the powder. The first material is sprayed on the substrate. The first material includes a plurality of different elements.

The second material can have one or more of the following features. The second material (e.g., tantalum, platinum, iridium, palladium, tungsten, gold, ruthenium, molybdenum, and/or rhenium) is more radiopaque than the first material. The second material is less radiopaque than the first material. The second material is wrapped around the first material.

In another aspect, the invention features a method of making a stent including delivering a first material to a substrate, melting the first material with a laser onto the substrate, solidifying the first material, delivering a second material to the substrate, melting the second material with the laser onto the first material, and solidifying the second material, wherein the first and second materials form a portion of the stent.

Embodiments may include one or more of the following features. The first and second materials define a tubular member. The method includes forming the tubular member into the stent. The second material more or less radiopaque than the first material. The method includes delivering a third material to the substrate, and melting the third material onto the second material. The third material is substantially the same as the first material.

In another aspect, the invention features a stent including a layer of material having an average of at least about nine grains per unit area. The layer can have an average of at least about twelve grains, e.g., at least about sixteen grains, per unit area. The layer can have a gradient of grain sizes along the thickness of the stent.

In another aspect, the invention features a stent including a layer of material having an average grain size less than about ten microns. The average grain size can be less than about eight microns, e.g., less than about six microns. The layer can have a gradient of grain sizes along the thickness of the stent.

In another aspect, the invention features a stent including a layer of material having a gradient of grain sizes along the thickness of the stent.

In yet another aspect, the invention features a stent including a layer of material having a gradient of composition along the thickness of the stent.

In another aspect, the invention features a method of making a medical device including delivering particles of a first material toward a substrate, applying energy to the particles to form a layer of the first material on the substrate, and using the layer to form the medical device.

Embodiments may include one or more of the following features. The energy is applied with a laser. The energy is applied simultaneously with delivery of the particles. The method includes melting at least a portion of the particles with the energy. The method includes changing the size of the particles.

In another aspect, the invention features a method including introducing a first material to a selected portion of a medical device, heating the first material or the selected portion, the first material and the selected portion fusing together.

Embodiments may include one or more of the following features. The first material is more radiopaque than a material of the medical device, and/or is capable of providing contrast during magnetic resonance imaging. The first material or the selected portion is heated with a laser. The first material is dispersed in a fluid. Heating is performed using magnetic induction. The selected portion includes a polymer. The medical device is a stent, and the selected portion is a tab extending from a portion of the stent. The first material and the selected portion fuse together to form an alloy. The medical device is a stent, a graft, a filter, a catheter, a guidewire, or an aneurysm coil.

Embodiments may have one or more of the following advantages. The endoprosthesis can be manufactured relatively fast and/or inexpensively. The endoprosthesis can be formed at a temperature range that result in relatively low interfacial diffusion or chemical segregation. Forming the endoprosthesis at relatively low temperatures can also reduce grain growth to provide a fine grain structure that strengthens the endoprosthesis. The endoprosthesis can be manufactured homogeneously, with accurate and precise compositions, e.g., relatively low elemental loss. Laser forming can eliminate the limitation of minimum metal charges and/or mold sizes, e.g., as possible with ingot casting. In some cases, mechanical processing (such as hot and/or cold working) or heating (e.g., annealing) to refine the grain structure can be reduced or eliminated.

The energy used in laser forming can be more focused and less than the energy used in certain types of deposition techniques, such as plasma deposition. The energy used in laser forming can also be applied sequentially, e.g., along a workpiece, during fabrication. The localized and sequential heating can result in less total heat input, which can reduce a heat-affected zone where grain growth can occur. As a result, a structure formed by laser forming can have relatively small and uniform grain size. The structure can also exhibit relatively homogeneous properties, e.g., relative to plasma spraying, sintering, or hot isostatic pressing in which the fabrication material is exposed to relatively deep or bulk heating and significant solid state diffusion is used to homogenize the material bulk properties. Using more controlled energy can also reduce residual stresses formed in the product, which can reduce (e.g., prevent) cracking or delamination between layers of materials. The focused energy from the laser allows fine patterns or detailing to be performed.

In some cases, laser forming can be used to form markers that enhance the fluoroscopic and/or MRI visibility of a medical device. The markers can be formed with strong adhesion to the device, without comprising the mechanical performance and/or dimensional profile of the device.

Other aspects, features, and advantages of the invention will be apparent from the description of the preferred embodiments thereof and from the claims.

### DESCRIPTION OF DRAWINGS

FIG. 1 illustrates a method of making an endoprosthesis.

FIG. 2 is a cross-sectional view of the endoprosthesis of FIG. 1, taken along line 2-2.

FIG. 3 is a schematic diagram of an embodiment of a laser forming system.

FIG. 4 is a schematic diagram of a portion of an embodiment of a laser forming system.

FIG. 5 is a schematic diagram of a portion of an embodiment of a laser forming system.

FIGS. 6A and 6B illustrate a portion of a method of making a medical device.

FIG. 7 is an illustration of a cross-section of a portion of an endoprosthesis.

FIG. 8 is an illustration of an embodiment of a tubular member.

FIG. 9 is an illustration of an embodiment of a tubular member.

FIG. 10 is an illustration of a portion of an endoprosthesis.

FIG. 11 is an illustration of a portion of an endoprosthesis.

FIG. 12 is an illustration of a portion of an endoprosthesis.

FIG. 13 is a schematic, cross-sectional view of an embodiment of a tubular member.

### DETAILED DESCRIPTION

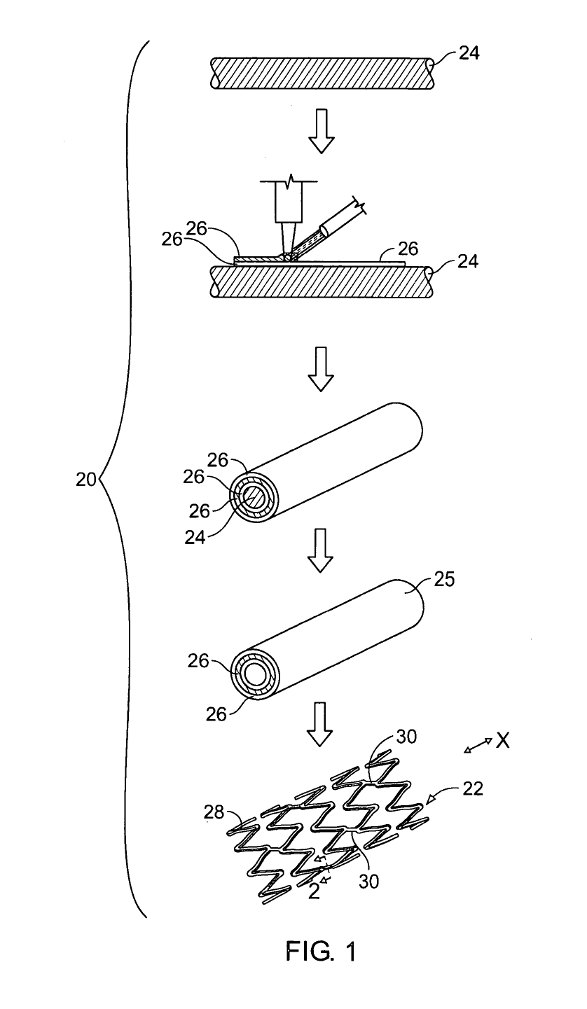

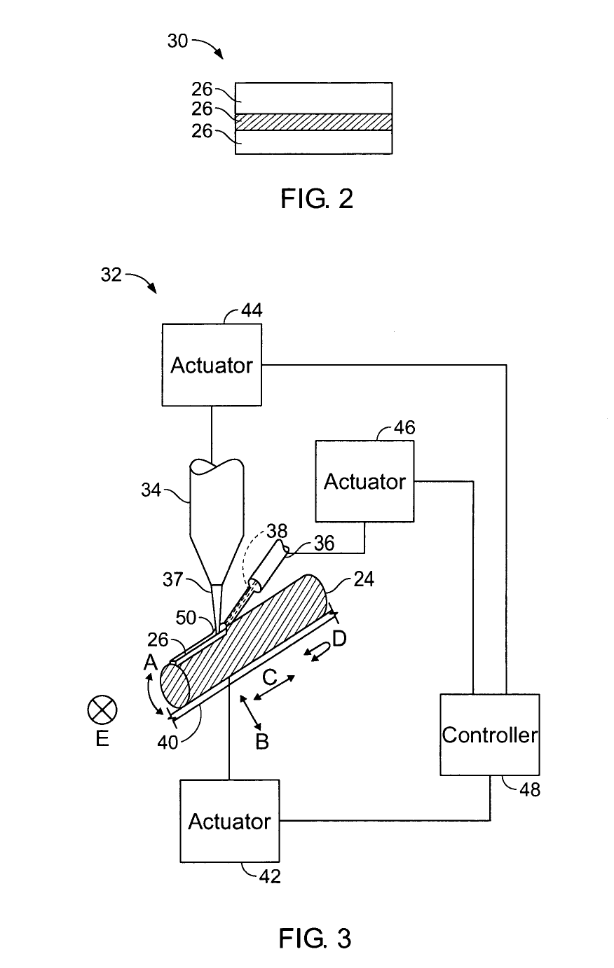

Referring to FIG. 1, a method 20 of making an endoprosthesis (as shown, a stent 22) is shown. Method 20 includes providing a substrate 24, such as a tubular mandrel, and forming on the substrate one or more layers 26 of material that ultimately define the structure of stent 22. In particular, layer(s) 26 are formed using a laser forming process that, as described herein, allows the microstructure, and therefore, the properties of stent 22 to be tailored, as well as providing other advantages. After layer(s) 26 are formed, substrate 24 is removed to yield a member 25 (as shown, a tubular member). Portions of layer(s) 26 are then removed, e.g., by laser cutting, to form stent 22 having bands 28 and struts 30 having one or more layers 26 (FIG. 2).

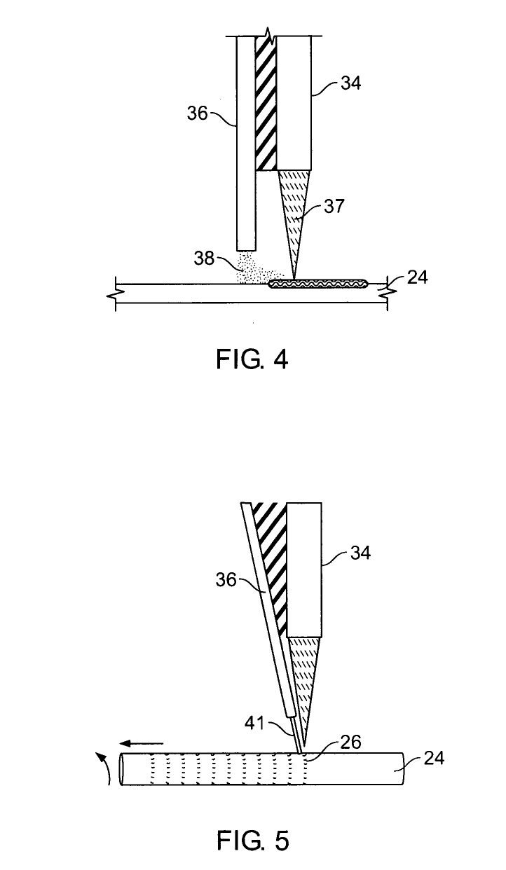

Referring to FIG. 3, the laser forming process includes a laser forming system 32. Laser forming system 32 includes a laser 34 and a feed nozzle 36. Feed nozzle 36 is capable of delivering the materials 38 of layer(s) 26, such as a powder, to substrate 24. Laser 34, e.g., a CO₂ laser, is capable of directing a laser beam 37 on substrate 24 to melt the materials delivered to the substrate by feed nozzle 36, thereby forming layers of material 38 on the substrate upon solidification. As shown, laser 34 and feed nozzle 36 can move independently of each other, but in other embodiments, referring to FIG. 4, the laser and the nozzle can be coupled together. Substrate 24 is positioned on a workstation 40 so that laser beam 37 and material 38 can be directed on a selected portion of the substrate. Workstation 40 can be, for example, a five-axis workstation that allows substrate 24 to be translated, rotated or otherwise maneuvered (e.g., arrows A, B, C, D, and E). Laser forming system 32 further includes actuators 42, 44, and 46 that control the activation and/or movement of workstation 40, laser 34, and feed nozzle 36, respectively. Actuators 42, 44, and 46 are interfaced to a controller 48 that is capable of processing input data, e.g., from a CAD file, and transmitting processed data to control the actuators, thereby controlling the operation of laser 34 and feed nozzle 36, and the orientation of substrate 24.

In operation, in response to the input data, controller 48 coordinates the movement and/or actuation of laser 34, feed nozzle 36, and substrate 24 such that material 38 is delivered, melted, and formed on selected areas of the substrate to form layer(s) 26. More specifically, during operation, feed nozzle 36 delivers material 38, e.g., powder, to laser beam 37, which heats the material and transforms the material, e.g., from solid particles to liquid or semi-liquid (i.e., partially liquid and partially solid) droplets. The droplets then land on substrate 24 and solidify or diffusion-bond. Since the mass of the particles is relatively small, the solidification rate can be fast such that deep pools of molten material are not present on the surface of substrate 24. Instead, there is a thin molten pool formed by reheated substrate material and the deposited liquid/semi-liquid material. The droplets can be heated somewhere above the solidus temperature such that they are semi-liquid; or the droplets can also be heated above the liquidus temperature such that they are liquid. Non-liquid material striking the surface of substrate 24 can bond to the substrate by elemental diffusion, and may require subsequent thermomechanical processing to strengthen the bonding. In some cases, the laser energy is sufficiently high that material 38 is melted, and there is melting of a thin layer of molten material on the substrate so that a layer is built can be formed by solidification.

Meanwhile, substrate 24 is maneuvered, e.g., translated, such that additional pools of molten material are formed on the substrate, e.g., adjacent to a previously formed pool. As previously formed pools are moved away from laser beam 37, the pools cool and solidify on substrate 24. By scanning or rastering substrate 24 across laser 34 and feed nozzle 36, or vice versa, strips of material 38 can be deposited on the substrate. The strips can be contiguously deposited to form a layer 26 surrounding substrate 24. The process described above can be repeated in a predetermined manner to form additional layer(s) on previously formed layers such that a strong metallurgical bond (e.g., welded or fused together) is formed between the layers. Portions of layer(s) 26 that are later removed (below) can be made thinner than portions that are not to be removed to reduce the amount the time and cost of forming stent 22. The compositions of the layers 26 can be varied, for example, by changing material 38 delivered through feed nozzle 36 and/or by changing the laser operating conditions.

Particular operating conditions for laser forming can be dependent, for example, on the materials used and the desired structure. In some cases, the laser can be a 100 W-10 kW CO₂ or Nd-YAG laser (for example, available from LASAG Industrial Lasers (Arlington Heights, Ill.)). An argon or helium carrier gas can be supplied at 50-300 psig and 5-30 liters/minute flow rate. An argon or helium shield gas can be supplied at 50-300 psig and 5-30 liters/minute flow rate. Material 38, such as a powder having a particle size of about 2-500 microns, can be delivered at 1-20 grams/minute. The stand-off distance (laser focus) can be about 0.10-200 mm. The focal spot size can be about 50-1000 microns in diameter. The traverse speed can be about 10-80 mm/second, and the deposition rate can be about 0.01-8 mm/second. The deposition thickness can be about 25-3000 microns.

Alternatively or in addition to the methods described above, other embodiments can be used. For example, referring to FIG. 5, feed nozzle 36 can be configured to deliver a wire or a filament 41 of material 38 to laser beam 37 for heating and melting. In other embodiments, multiple feed nozzles can be used, e.g., to form an alloy composition. Alternatively or in addition, an alloy powder can be pre-synthesized and delivered through one or more feed nozzles.

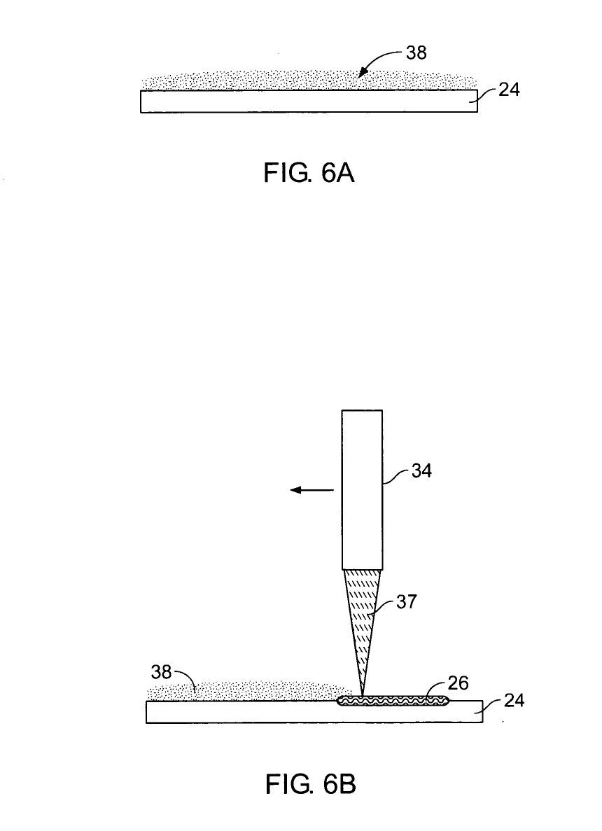

In some embodiments, no feed nozzle is used. Referring to FIGS. 6A and 6B, material 38 can be dispersed in a liquid carrier, and the dispersion can be applied to substrate 24. The carrier can then be removed to leave a coating of material 38 adhered on substrate 24, and subsequently, laser 34 can be used to heat and melt the material to form layer 26 as described above. The liquid carrier can be a high vapor pressure liquid, such as an organic solvent (e.g., an alcohol), which can be removed gentle heating. The dispersion can be applied to substrate by coating, dipping, spraying, and/or brushing. Material 38 can be a pre-synthesized final product or a mixture of the components of the final product.

Laser forming processes, including exemplary laser forming systems, can be found, for example, in Pyritz et al., U.S. Pat. No. 6,396,025; Kobryn et al., Scripta Mater. 43 (2000) 299-205; Kahlen et al., J. Laser Anpl., Vol. 13, No. 2, April 2001, 60-69; and ASM Handbook Volume 5. Laser forming processes can be performed by vendors, such as AeroMet Corporation (Eden Prairie, Minn.), which markets its services as Lasform^{SM}.

As indicated above, the laser forming process allows stent 22 to be formed with a selected microstructure. For example, stent 22 can be form with a polycrystalline structure having relatively small or fine grains. The grains can be long and thin as a result of molten or semi-molten droplets striking the substrate during deposition. The fine grain structure can strengthen stent 22 by providing tortuous paths extending throughout the structure that reduce (e.g., inhibit) crack propagation. The fine grain structure can also provide stent 22 with relatively uniform, homogeneous properties. In comparison, large, coarse grains are more likely to preferentially orient, e.g., in relation to a tensile axis, such that certain slip systems are activated, which can lead to delamination and/or spoiling. Local areas having coarse grain size relative to a cross-sectional distance of the stent (e.g., one or two grains across the thickness of a stent strut) can neck down to a thin, knife edge if the stent is overly expanded and fractures. The sharp edge can cause trauma to a vessel wall during use.

In some embodiments, stent 22 has at least nine grains per unit area. For example, per unit area, stent 22 can have at least twelve grains, at least sixteen grains, at least 20 grains, at least 25 grains, at least 36 grains, or higher. As used herein, a unit area is the square of the thickness of a layer of the stent. The number of grains is an average number of grains taken over a substantial number (e.g., 20 or more) of cross sections of the stent. Preferably, the number of grains per unit area is uniformly distributed throughout the entire stent. The size of the grains can be changed by changing the laser forming conditions. For example, to form smaller grains, smaller powder (e.g., on the order of microns) can be used, and/or the power of the laser can be adjusted to change the degree of heating and melting.

Alternatively or in addition, the fine grain structure of stent 22 can be expressed in terms of an average grain size (e.g., diameter). Table 1 shows how the average grain size (diameter) for four stent wall thicknesses (1-4 mil) can be related to the number of grains per unit area.

TABLE 1

| | Wall | | Grain/ | | |
|---|---|---|---|---|---|
| Wall | thickness | Unit Area | Unit Area | | Avg. Grain |
| thickness | (T) | (T²) | (9/T², | ASTM | Diameter |
| (inch) | (mm) | (mm²) | grain/mm²) | E112 G | (microns) |
| 0.001 | 0.025 | 0.0006 | 13950 | >14 | 2 |
| 0.002 | 0.051 | 0.0026 | 3488 | 12.5 | 4.7 |
| 0.003 | 0.076 | 0.0103 | 1550 | 11.5 | 6.7 |
| 0.004 | 0.102 | 0.161 | 872 | 10.5 | 9.4 |

As indicated above, the unit area is determined by squaring the thickness of a layer of the stent, e.g., the thickness of a layer of a stent strut. The number of grains per unit area (in this example, nine grains/unit area) can then be converted to an ASTM E112 G value. (See ASTM E112 Table 4. Grain Size Relationships Computed for Uniform, Randomly Oriented, Equiaxed Grains.) The average grain diameter can then be determined from ASTM E112 G value, which is inversely proportional to the average grain diameter. (See ASTM E112.) In some embodiments, stent 22 has an average grain diameter of less than about ten microns. For example, the average grain diameter can be equal to or less than about nine microns, eight microns, seven microns, six microns, five microns, four microns, three microns, two microns, or one micron; and/or greater than or equal to one micron, two microns, three microns, four microns, five microns, six microns, seven microns, eight microns, or nine microns.

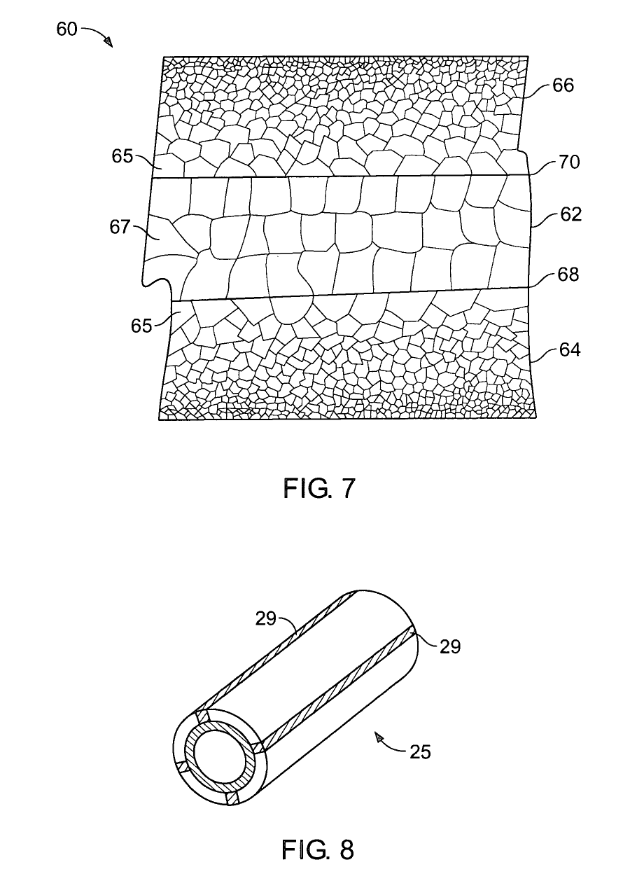

In certain cases, the grain structure (e.g., size) within a layer can vary. For example, referring to FIG. 7, a cross section of a stent 60 includes an intermediate layer 62 (e.g., a radiopaque layer including tantalum) formed between a first layer 64 and a second layer 66 (e.g., inner and outer layers including a structural material such as stainless steel). The grain structures of layers 64 and 66 vary along the thickness of stent 60. In particular, at the interfaces 68 and 70 between intermediate layer 62 and layers 64 and 66, respectively, the size of grains 65 in layers 64 and 66 substantially matches the size of grains 67 in the intermediate layer. The well-matched interfaces reduce interfacial differences in physical properties (e.g., thermal conductivity or magnetic susceptibility) and/or mechanical properties (e.g., strength or ductility). The interfacial differences can result in sub-optimal bonding between the layers and/or failure at the interfaces can occur during use or during manufacture.

In addition, with increasing distance from interfaces 68 and 70, the size of grains in layers 64 and 66 decreases to provide a fine grain structure. As an example, the size of grains in layers 64 and 66 can vary from about six microns to about fifteen microns at the interfaces. The gradient of grain size can also provide a balance of mechanical properties since a fine grain structure can provide good strength, while a coarse grain structure can provide good ductility. The size of the grains can be changed as described above.

The composition within a layer can also be varied. The composition can be changed to affect the mechanical and physical properties of the stent, and/or to reduce a sharp transition between different layers, which can affect the bonding between the layers. For example, layer 64 can be formed starting with a structural material (such as stainless steel). As the thickness of layer 64 approaches interface 68, a compositional gradient can be formed, e.g., by increasing the amount of a radiopaque material such as tantalum to the stainless steel. As a result, when intermediate layer 62 is formed (e.g., from tantalum), there is less of a difference in compositions at interface 68 between layers 62 and 64. The composition within a layer can be adjusted, for example, by changing the materials delivered through the feed nozzle.

In addition to being able to form a stent with predetermined microstructure, the laser forming process also allows the stent to be designed with preselected configurations of layers. By selecting the appropriate materials and forming (e.g., layering or grading) the materials in a predetermined configuration, the stent can have preselected mechanical, physical, or chemical properties. Generally, stent 22 includes one or more portions that provide the stent with strength, ductility, stiffness, density, and biocompatibility. Stent 22 can also include one or more materials selected and formed to provide the stent with a preselected radiopacity and/or MRI visibility so that the stent can be tracked and monitored.

For example, for a balloon expandable stent, the stent can include one or more materials with preselected mechanical properties so that the stent can be compacted, and subsequently expanded with relatively easy plastic flow during balloon expansion. The stent preferably has good resistance to recoil and radial compression after balloon expansion. As one model, a balloon expandable stent can be formed of annealed 316L stainless steel. The stent can have an ultimate tensile strength (UTS) of about 70-100 ksi, greater than about 25% elongation to failure, and a modulus of elasticity of about 26 msi. When the stent is expanded, the material is stretched to strains on the order of about 0.3. The ultimate tensile strength of the stretched 316L stainless steel is estimated to increase to about 140-160 ksi, and the elongation is estimated to drop to about 10%. In some cases, finite element analysis (FEA) models can be used to design the mechanical properties of a stent. Suitable “structural” materials that provide good mechanical properties and/or biocompatibility include, for example, stainless steel (e.g., 316L stainless steel), Nitinol (a nickel-titanium alloy), Elgiloy, L605 alloys, Ti-6A1-4V, and Co-28Cr-6Mo. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; PCT application US91/02420; and commonly assigned U.S. Ser. No. 10/346,487, filed Jan. 17, 2003.

Stent 22 can also include one or more layers of radiopaque material to provide radiopacity. Suitable radiopaque materials include metallic elements having atomic numbers greater than 26, e.g., greater than 43. In some cases, the materials have a density greater than about 9.9 g/cc. In certain embodiments, the radiopaque material is relatively absorptive of X-rays, e.g., having a linear attenuation coefficient of at least 25 cm⁻¹, e.g., at least 50 cm⁻¹, at 100 keV. Some radiopaque materials include tantalum, platinum, iridium, palladium, hafnium, tungsten, gold, ruthenium, and rhenium. The radiopaque material can include an alloy, such as a binary, a ternary or more complex alloy, containing one or more elements listed above with one or more other elements such as iron, nickel, cobalt, or titanium. A mixture (e.g., a powder mixture) of radiopaque material(s) and structural material(s) can be delivered to and formed at selected portion(s) of stent 22, e.g., at its ends. The mixture can enhance radiopacity without adversely affecting the mechanical properties of the stent. In some cases, the radiopaque material does not contribute substantially to the mechanical properties of the stent, but by forming the radiopaque material with a microstructure similar to that of the structural material, the difference in properties and stress concentration (which can lead to shearing) can be reduced (e.g., minimized) to provide a homogeneous composite. Example 1 provided below illustrates some considerations in designing the radiopacity of a stent.

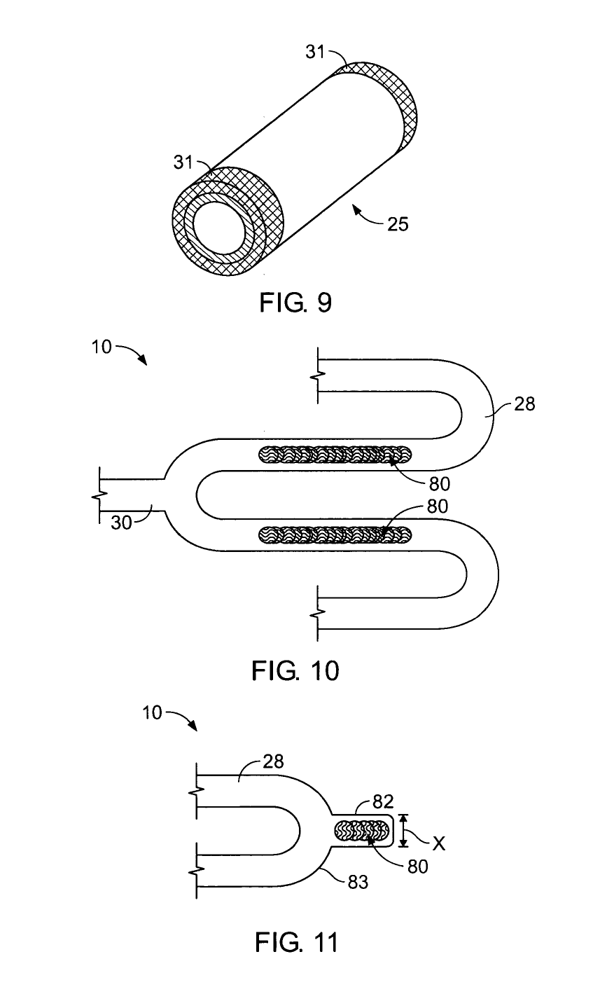

Stent 22 generally includes one or more layers. A one-layer stent can include, e.g., a middle body portion including one or more structural materials, and end portions including a radiopaque material(s), or a mixture of structural material(s) and radiopaque material(s). An entire layer can include structural material(s) and radiopaque material(s), e.g., as described in U.S. Ser. No. 10/338,223, filed Jan. 8, 2003. A two-layered stent can include a radiopaque layer and a structural layer. Either layer can be the inner or the outer layer. A three-layered stent can include a radiopaque layer formed between two structural layers. A layer can include one or more materials (FIG. 8). A layer can partially extend radially around member 25, and/or partially along the length of the member. For example, a layer can be formed having stripes (e.g., longitudinally extending stripes 29) of radiopaque material and/or structural material (FIG. 8). A layer can be formed as rings 31 at selected portions (e.g., at the ends) of member 25 (FIG. 9).

Referring again to method 20 shown in FIG. 1, after layer(s) 26 are formed, substrate 24 is removed to yield member 25. Substrate 24 can be dissolved, e.g., a carbon steel substrate can be dissolved by immersion in an acid such as nitric acid, which can also remove certain recast material formed during manufacturing. Alternatively or in addition, substrate 24 can be mechanically removed (e.g., by grinding), melted (e.g., for materials having sufficiently low melting points), and/or sublimed. In some cases, the material(s) for substrate 24 can react with layer(s) 26 to form a product that is convenient or easy to remove. Substrate 24 can have similar or higher melting points as that of layer(s) 26, e.g., so that the substrate 24 and the layer(s) can be annealed with the tubular member, i.e., no melting or degradation during heating. Examples of materials for substrate 24 include metallic materials, such as carbon steel, cadmium, lead, magnesium, tin, zinc, titanium, stainless steel (e.g., 304L or 316L stainless steel), and aluminum. In some embodiments, substrate 24 includes a ceramic and/or glass. Substrate 24 can be removed by heating the substrate and allowing differential thermal expansion to separate the substrate from layer(s) 26. Alternatively or in addition, heating and rapid quenching can be used to shatter a brittle substrate for removal.

In some embodiments, member 25 can be mechanically worked, before or after substrate 24 is removed. For example, member 25 can drawn to reduce the size of the member. Member 25 can also be cold worked and/or heated (e.g., annealed, hot isostatically pressed, and/or recrystallized) to change the grain structure. Such processing procedures can produce an equiaxed grain morphology similar to the grain structure from powder metallurgy techniques.

After substrate 24 is removed, selected portions of layer(s) 26 are removed to form the structure (e.g., openings 28 and struts 30) of stent 22. The portions can be removed by laser cutting, as described in U.S. Pat. No. 5,780,807, hereby incorporated by reference in its entirety. In certain embodiments, during laser cutting, a liquid carrier, such as a solvent or an oil, is flowed through member 25 (arrow X). The carrier can prevent dross formed on one portion of member 25 from re-depositing on another portion, and/or reduce formation of recast material on the tubular member. Other methods of removing portions of layer(s) 26 can be used, such as mechanical machining (e.g., micro-machining), electrical discharge machining (EDM), and photoetching (e.g., acid photoetching). In certain embodiments, selected portions of layer(s) 26 are removed before substrate 24 is removed.

Stent 22 can then be finished, e.g., electropolished to a smooth finish, according to conventional methods. In some cases, since member 25 can be formed to near-net size, relatively little of the member need to be removed to finish the stent. “Near-net size” means that member 25 has a relatively thin envelope of material that is removed to provide a finished stent. In some cases, member 25 is formed less than about 25% oversized, e.g., less than about 15%, 10%, or 5% oversized. As a result, further processing (which can damage the stent) and costly materials can be reduced. In some embodiments, about 0.0001 inch of the stent material can be removed from each surface by chemical milling and electropolishing to yield a stent. Stent 22 can then be annealed.

Throughout the making of stent 22, the stent or member 25 can be pressed (e.g., mechanically or hot isostatically treated) and/or heated (e.g., sintered, age hardened, or annealed). The pressing and/or heating can change the physical properties (e.g., porosity) and/or the mechanical properties (e.g., strength) of the stent or the member.

In use, stent 22 can be used, e.g., delivered and expanded, according to conventional methods. Suitable catheter systems are described in, for example, Wang U.S. Pat. No. 5,195,969, and Hamlin U.S. Pat. No. 5,270,086. Suitable stents and stent delivery are also exemplified by the Radius® or Symbiot® systems, available from Boston Scientific Scimed, Maple Grove, Minn.

Generally, stent 22 can be of any desired shape and size (e.g., coronary stents, aortic stents, peripheral vascular stents, gastrointestinal stents, urology stents, and neurology stents). Depending on the application, stent 22 can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stent 22 can be balloon-expandable, self-expandable, or a combination of both (e.g., U.S. Pat. No. 5,366,504).

Stent 22 can also be a part of a stent-graft. In other embodiments, stent 22 can include and/or be attached to a biocompatible, non-porous or semi-porous polymer matrix made of polytetrafluoroethylene (PTFE), expanded PTFE, polyethylene, urethane, or polypropylene. The endoprosthesis can include a releasable therapeutic agent, drug, or a pharmaceutically active compound, such as described in U.S. Pat. No. 5,674,242, U.S. Ser. No. 09/895,415, filed Jul. 2, 2001, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

In some embodiments, layer(s) 26 includes one or more materials that enhance visibility by magnetic resonance imaging (MRI). Examples of MRI visible materials include non-ferrous metal-alloys containing paramagnetic elements (e.g., dysprosium or gadolinium) such as terbium-dysprosium, dysprosium, and gadolinium; non-ferrous metallic bands coated with an oxide or a carbide layer of dysprosium or gadolinium (e.g., Dy₂O_{3 or Gd}₂O₃); non-ferrous metals (e.g., copper, silver, platinum, or gold) coated with a layer of superparamagnetic material, such as nanocrystalline Fe₃O₄, CoFe₂O₄, MnFe₂O₄, or MgFe₂O₄; and nanocrystalline particles of the transition metal oxides (e.g., oxides of Fe, Co, Ni). Alternatively or in addition, layer(s) 26 can include one or more materials having low magnetic susceptibility to reduce magnetic susceptibility artifacts, which during imaging can interfere with imaging of tissue, e.g., adjacent to and/or surrounding the stent. Low magnetic susceptibility materials include tantalum, platinum, titanium, niobium, copper, and alloys containing these elements.

Laser forming can be used to form hollow structures that can be subsequently filled. For example, the laser forming process can be used to form a member having one or more removable core portions (e.g., carbon steel or ceramics) surrounded by portions of structural material and/or radiopaque material. After the portions of structural material and/or radiopaque material are formed, the substrate and the core portion can be removed, e.g., by dissolution in acid or other solutions, to reveal one or more passageways. The passageway(s) can be filled with one or more selected materials, such as radiopaque materials, radioactive materials (e.g., for brachytherapy), or drugs. Such techniques are similar to investment casting.

In other embodiments, alternatively or in addition to forming a radiopaque layer, a strip or wire of radiopaque material can be used. For example, to form a tubular member, a first layer (e.g., 316L SS) can be formed on a substrate by laser forming. Next, a strip or a wire of radiopaque material (e.g., tantalum) can be wound, woven, or braided over the first layer. A second layer (e.g., 316L SS) can be laser formed over the radiopaque strip or wire. The formed tubular member can be formed into a stent as described above.

In some embodiments, the laser forming process can be used to form a non-tubular member, e.g., a sheet. For example, the formed sheet can be further mechanically worked (e.g., drawn, forged, rolled, and/or extruded) and seam welded to form a tubular member. The formed tubular member can be formed into a stent as described above.

In other embodiments, the processes described herein can be used to make other medical devices. Such devices include surgical tools or implants, e.g., hip implants or tibial trays. Other devices include guidewires (such as a Meier steerable guide wire (for AAA stent procedure) and an ASAP Automated Biopsy System, e.g., described in U.S. Pat. Nos. 4,958,625, 5,368,045, and 5,090,419); filters (such as removable thrombus filters, e.g., described in U.S. Pat. No. 6,146,404, intravascular filters, e.g., described in U.S. Pat. No. 6,171,327, and vena cava filters, e.g., described in U.S. Pat. No. 6,342,062); markers bands; aneurysm or vaso-occlusive coils; and catheter components, e.g., hypotube catheters.

The processes described herein can also be used to enhance a medical device, such as the embodiments described herein or other pre-fabricated devices. For example, referring to FIG. 10, laser forming techniques can be used to enhance the radiopacity and/or MRI visibility of one or more selected portions of a stent. As shown, band 28 of stent 10 includes two portions 80 including a radiopaque material and/or a material capable of enhancing MRI visibility of the stent. In particular, portions 80 are local areas or zones having a radiopaque material and/or an MRI visibility enhancing material fused into stent 10. In some cases, portions 80, which are integrally formed on the surface of stent 10, include an alloy of the base stent material (e.g., stainless steel or a titanium alloy such as Nitinol) and the radiopaque material and/or the MRI visibility enhancing material.

Portions 80 can be made by any of the processes described herein, for example, illustrated in FIGS. 3, 4, 5, and 6A-6B (e.g., with suitable masking). Changing the laser power (hence, degree of heating and melting) and the amount of material introduced can be used to control the thickness of portions 80. The thickness of portions 80 can be a function of, for example, the number of portions formed on the medical device, the type of medical device, other dimensions of the portions, the material(s) in the portions, and the targeted visibility. For example, for a peripheral vascular or coronary stent, the thickness of portion 80 can be from about 2 to about 20 microns, e.g., from about 5 to about 10 microns. Thicker portions 80 in the range of from about 20 to about 200 microns may be used for larger stents used to treat vessels such as the aorta, esophagus or lower digestive tract. In some cases, the thickness of portions 80 is from about 12 microns to about 50 microns for stents with wall thicknesses of about 25 microns to about 150 microns, and from about 25 microns to about 125 microns for stents with wall thicknesses of about 175 microns to about 375 microns. Portions 80 can be formed in areas subjected to low levels of deformation or strain during loading and deployment and low cyclic deformation when implanted in the body. Examples of areas include the long segment length between nodes (e.g., as shown in FIG. 10) and any tabs that extend off of the main strut structure and do not contribute significantly to the structure of the device. The processes described herein are capable of providing markers that enhance the visibility of the medical device, without compromising the performance or dimensional profile of the device.

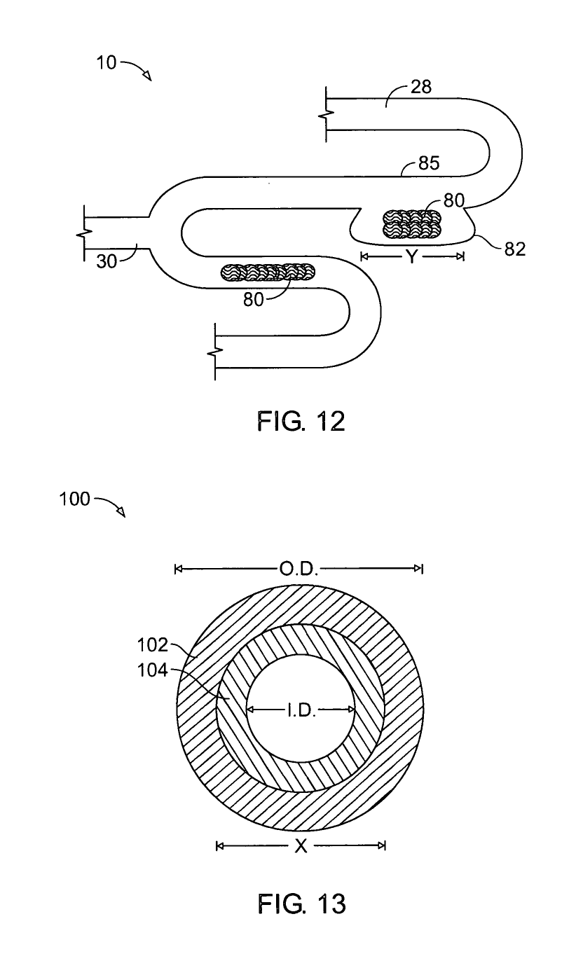

Referring to FIGS. 11 and 12, in some embodiments, to reduce any adverse effect of portions 80 on the performance of stent 10 (e.g., its expansion, crimping, or mechanical properties), the stent includes one or more extensions or tabs 82 on which portions 80 are formed. Tab 82 can extend from an end portion 83 and/or from a side portion 85 of band 28 and generally parallel to the longitudinal axis of stent 10. Tab 82 can be of any shape, for example, including a rectangular, oval, circular, or parallelogram portion, and be regular or irregular. As shown in FIG. 11, tab 82 can be attached to stent 10 along its short dimension (X), or as shown in FIG. 12, the tab can be attached to the stent along its long dimension (Y). In some cases, tab 82 includes an opening through which a piece (e.g., a plug or a disc) of radiopaque material and/or MRI visible material can be placed. The piece of material can be fused to tab 82 using laser heating, tungsten inert gas (TIG) welding, plasma welding, resistance welding, and/or electron beam welding.

One or more portions 80 can be formed on any of the medical devices (e.g., aneurysm coils, filters, guidewires, or catheters) describe above.

In some cases, the medical devices are fabricated from a polymer. The radiopaque material and/or the MRI visible material can be applied to the device, and subsequently, thermal energy (e.g., from a laser) can be applied to melt the polymer surface and allow the radiopaque material and/or the MRI visible material to fuse with the polymer, thereby enhancing the visibility of the device. Alternatively or in addition, magnetic induction (e.g., U.S. Pat. No. 6,056,844) can be used to heat the radiopaque material and/or the MRI visible material to the melting point of the polymer, thereby locally melting the polymer and allowing the material(s) to fuse with the polymer. Examples of polymer medical devices include polymer stents (e.g., U.S. Ser. No. 10/229,548, filed Aug. 28, 2002; U.S. Ser. No. 60/418,023, filed Oct. 11, 2002); polymer catheters; polymer guidewires (e.g., U.S. Pat. No. 6,436,056); filters; and vascular grafts (e.g., U.S. Pat. No. 5,320,100).

The following example is illustrative and not intended to be limiting.

### EXAMPLE 1

The following example illustrates a method of designing the radiopacity of a stent.

A good level of radiopacity is one where the stent can be seen, but where the stent image is not so bright as to obscure tissue and flow of fluid around and through the stent. The density of 316L stainless steel is about 8.0 g/cc. It is estimated that for small diameter, thin wall stents, the density can be about 40% higher than 316L stainless steel to provide good radiopacity. For an upper limit, the radiopacity can be limited to be 10% lower than the density of tantalum (16.6 g/cc), or about 14.9 g/cc, because a stent made of tantalum can be too bright in a fluoroscopic image.

FIG. 13 shows a two-layer tubular member 100 having an outer layer 102 including 316L stainless steel and an inner layer 104 including tantalum. As an example, tubular member 100 has an outer diameter (O.D.) of 0.203 cm and an inner diameter (I.D.) of 0.183 cm. To calculate diameter X, and thus the concentration of 316L stainless steel and tantalum in tubular member 100, the mass (M) of the tubular member (1 inch long) is first calculated:
M=ρ_{goal}[π/4 (O.D.²−I.D.²)] (1)
where ρ_{goal} is a targeted density, e.g., 11.2 g/cc. In this example, the mass (M) is 0.173 g. Diameter X can then be calculated using a mass balance calculation:
M=ρ_{IL}[π/4 (O.D.²−X²)]+ρ_{OL}[π/4(X²−I.D.²)] (2)
where ρ_{IL} is the density of the inner layer (e.g., 8.0 g/cc for 316L SS), and ρ_{OL} is the density of inner layer (e.g., 16.6 g/cc for Ta). Solving Equation (2) for X yields X=0.191 cm.

The same method of calculation can be used to design a tubular member having the radiopaque material in the inner layer and stainless steel in the outer layer. The stainless steel inner layer can allow the inner surface of the stent to be electropolished and cleaned to produce a surface finish with good thrombogenicity and blood flow characteristics. Also, having the relatively more radiopaque material in the outer layer can result in more attenuation of X-rays and more contrast in the radiographic image between the stent the surround tissue and/or bone.

The same method of calculation can be adapted to design a tubular member having more than two layers, e.g., a radiopaque layer between two stainless steel layers.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

All publications, references, applications, and patents referred to herein are incorporated by reference in their entirety.

Other embodiments are within the claims.

1. A method of making a medical device, comprising:
melting a first material on a substrate; and
solidifying the material,
wherein the solidified first material forms a portion of the medical device.

2. The method of claim 1, wherein the first material is melted by a laser.

3. The method of claim 2, further comprising moving the laser or the substrate relative to each other.

4. The method of claim 1, further comprising forming a layer of the first material surrounding the substrate.

5. The method of claim 1, further comprising melting a second material on the first material.

6. The method of claim 5, wherein the second material is more radiopaque than the first material.

7. The method of claim 6, wherein the second material comprises a material selected from the group consisting of tantalum, platinum, iridium, palladium, tungsten, gold, ruthenium, molybdenum, and rhenium.

8. The method of claim 6, wherein the first material comprises stainless steel.

9. The method of claim 5, wherein the second material is less radiopaque than the first material.

10. The method of claim 1, further comprising delivering the first material to the substrate.

11. The method of claim 10, wherein the first material is in the form of a powder.

12. The method of claim 1, further comprising changing the size of the powder.

13. The method of claim 10, wherein the first material is sprayed on the substrate.

14. The method of claim 1, wherein the first material comprises a plurality of different elements.

15. The method of claim 1, further comprising separating a portion of the substrate from the first material.

16. The method of claim 1, wherein the first material is in the form of a powder.

17. The method of claim 1, comprising forming the first material into a tubular member.

18. The method of claim 17, further comprising forming the tubular member into a stent.

19. The method of claim 1, wherein the first material comprises an alloy.

20. The method of claim 1, further comprising changing the composition of the first material.

21. The method of claim 1, wherein the first material is melted by a laser, and further comprising changing the operating conditions of the laser during melting of the first material.

22. The method of claim 1, further comprising placing an elongated second material more radiopaque than the first material on the solidified first material.

23. The method of claim 1, wherein the second material is wrapped around the first material.

24. A method of making a stent, comprising:
delivering a first material to a substrate;
melting the first material with a laser onto the substrate;
solidifying the first material;
delivering a second material to the substrate;
melting the second material with the laser onto the first material; and
solidifying the second material,
wherein the first and second materials form a portion of the stent.

25. The method of claim 24, wherein the first and second materials define a tubular member.

26. The method of claim 25, further comprising forming the tubular member into the stent.

27. The method of claim 24, wherein the second material more radiopaque than the first material.

28. The method of claim 24, wherein the second material is less radiopaque than the first material.

29. The method of claim 24, further comprising delivering a third material to the substrate, and melting the third material onto the second material.

30. The method of claim 29, wherein the third material is substantially the same as the first material.

31. A stent, comprising a layer of material having an average of at least about nine grains per unit area.

32. The stent of claim 31, wherein the layer has an average of at least about twelve grains per unit area.

33. The stent of claim 31, wherein the layer has an average of at least about sixteen grains per unit area.

34. The stent of claim 31, wherein the layer has a gradient of grain sizes along the thickness of the stent.

35. A stent, comprising a layer of material having an average grain size less than about ten microns.

36. The stent of claim 35, wherein the average grain size is less than about eight microns.

37. The stent of claim 35, wherein the average grain size is less than about six microns.

38. The stent of claim 35, wherein the layer has a gradient of grain sizes along the thickness of the stent.

39. A stent, comprising a layer of material having a gradient of grain sizes along the thickness of the stent.

40. A stent, comprising a layer of material having a gradient of composition along the thickness of the stent.

41. A method of making a medical device, comprising:
delivering particles of a first material toward a substrate;
applying energy to the particles to form a layer of the first material on the substrate; and
using the layer to form the medical device.

42. The method of claim 41, wherein the energy is applied with a laser.

43. The method of claim 42, wherein the energy is applied simultaneously with delivery of the particles.

44. The method of claim 41, comprising melting at least a portion of the particles with the energy.

45. The method of claim 41, comprising changing the size of the particles.

46. A method, comprising:
introducing a first material to a selected portion of a medical device;
heating the first material or the selected portion, the first material and the selected portion fusing together.

47. The method of claim 46, wherein the first material is more radiopaque than a material of the medical device.

48. The method of claim 46, wherein the first material is capable of providing contrast during magnetic resonance imaging.

49. The method of claim 46, wherein the first material or the selected portion is heated with a laser.

50. The method of claim 46, wherein the first material is dispersed in a fluid.

51. The method of claim 46, wherein the heating is performed using magnetic induction.

52. The method of claim 46, wherein the selected portion comprises a polymer.

53. The method of claim 46, wherein the medical device is a stent, and the selected portion is a tab extending from a portion of the stent.

54. The method of claim 46, wherein the first material and the selected portion fuse together to form an alloy.

55. The method of claim 46, wherein the medical device is selected from the group consisting of a stent, a graft, a filter, a catheter, a guidewire, and an aneurysm coil.
